(19) United States
(12) Patent Application Publication
(10) Pub. No.: US 2023/0310062 A1
(43) Pub. Date: Oct. 5, 2023

(54) ENDOSCOPIC SURGERY LEARNING ASSISTANCE METHOD AND SYSTEM

(71) Applicants: UNIVERSITE DE LORRAINE, Nancy (FR); CENTRE HOSPITALIER REGIONAL UNIVERSITAIRE DE NANCY, Nancy (FR)

(72) Inventors: Patrice GALLET, Villers-les-Nancy (FR); Valentin FAVIER, Vailhauques (FR); Guillaume CINI, Lenoncourt (FR); Cécile RUMEAU, Pulnoy (FR)

(21) Appl. No.: 18/004,783

(22) PCT Filed: Jul. 8, 2021
(86) PCT No.: PCT/EP2021/069074

§ 371 Date: Jan. 9, 2023

(30) Foreign Application Priority Data
Jul. 10, 2020 (FR) 2007361

(51) Int. Cl. A61B 18/14; A61B 17/29

(52) U.S. Cl. CPC A61B 18/1445; A61B 17/29; A61B 2017/2927

(57) ABSTRACT

A method and system for assisting learning of endoscopic surgery, in particular endonasal surgery, during exercises each including a set of instrument movements of the endoscope and/or of one or more surgical instruments, carried out by the operator. The system includes a hardware simulator including one or more thematic models, in particular interchangeable modules on a support head. It also includes a computerised management unit which communicates with the endoscope and/or the instrument and/or the module or model. For each exercise, it displays the actions to be performed, and detects various endoscope and/or instrument movements and/or contacts. One module presents targets to be visualised in a framed manner, the display of which is validated by shape recognition in the software. Other modules include objects to be dissected or moved, while avoiding obstacles. The movements of instruments, grippers and/or contacts, are recorded by the software in order to evaluate the exercise.

The invention relates to a method and system for assisting the learning of endoscopic, in particular endonasal, surgery during exercises each comprising a set of instrument movements, of the endoscope and/or of one or more surgical instruments, carried out by the operator.

The system comprises a hardware simulator including one or more thematic models, in particular interchangeable modules on a support head.

It also comprises a computerized management unit which communicates with the endoscope and/or the instrument and/or the module or model. For each exercise, it displays the actions to be carried out, and detects different movements and/or contacts, of the endoscope and/or instrument.

One module presents targets to be visualized in a framed manner, the display of which is validated by shape recognition in the software. Other modules comprise objects to be dissected or moved, while avoiding obstacles. The prohibited or requested movements of instruments, forceps, and/or contacts are recorded by the software in order to evaluate the exercise.

### STATE OF THE ART

A former practice for learning the moves of, human or veterinary, surgery, whether in the instruction or improvement or training phase, was to use cadavers. For reasons of simplicity and hygiene, it is now known to train using manikins, which realistically reproduce the region of the body concerned, in terms of shape and colour and sometimes texture.

In the case of endoscopic surgery, the hardware context of the moves is quite specific, for example because it is a closed environment and a restricted space. The instruments used are adapted to this context, and are themselves quite specific in their structure and their use. These instruments in most cases comprise two different elements which can be manipulated independently of each other, whether they are totally distinct instruments or separate from each other at the distal end of a common body. One of these instruments is the surgical instrument, generally manipulated by the directing hand. At its distal end, it carries a working head which serves to carry out the hardware interaction with the tissues concerned, for example a dissection with bone-cutting forceps, or an incision with a blade, or a suture by gripping and moving a needle by means of simple forceps. The other instrument is the visualization instrument, or endoscope, often manipulated by the non-directing hand. At its distal end, it carries a lens system which has to be moved closer to and oriented towards the working area, generally equipped with lighting. This lens system is connected to a visualization device situated at the proximal end, for example a screen or an eyepiece, which makes it possible for the operator to visualize the image captured by the lens system, and thus to see the area to be treated and see the action of their surgical instrument. It is also possible for this surgery to be carried out by several operators simultaneously, “with four hands”, each using their own surgical instrument.

An example of regularly practiced endoscopic surgery is endonasal surgery, which relates to the interior of the nostrils or nasal cavities, or even the sinuses.

For example, but not exclusively, above all for shallow operations, totally distinct instruments are used, which are introduced independently of each other.

In the case of deeper operations, the endoscope often has a longitudinal “operating” channel in which the surgical instrument is inserted, which can thus be easily extracted and replaced during the operation in order to use several types of instrument one after another.

For example for endonasal surgery, it is known to produce a model, or “phantom”, which simulates the nasal cavity and contains an artificial but realistic element which reproduces a portion of the anatomy on which the operator is learning to operate, for example a polyp to be removed or a septum to be incised or dissected. The document EP1619644presents for example a phantom head, which realistically reproduces the region of the paranasal sinus, and includes a destructible portion which can be replaced in order to repeat the exercise.

However, such a learning process remains a long and tricky process, including a large number of abilities to be acquired; for which such a manikin can present a complexity and a substantial cost, and the teaching and supervision of which are difficult and time- and energy-consuming.

An aim of the invention is to overcome, in full or in part, the drawbacks of the state of the art. It is sought in particular to make a better flexibility possible in the progress of such a learning process, to make it easier for the learner to focus, to make it easier to adapt the hardware and the teacher to the learner's progress, and to make precise and specific feedback on the carrying-out of one or more moves possible.

### PRESENTATION OF THE INVENTION

The invention proposes a method for assisting the learning, by an operator, of endoscopic surgery applied to at least one region of interest of the human or animal body, in particular an endonasal region, during an exercise carried out by said operator.

This exercise comprises a set of movements of instruments operated by hand, carried out by said operator, by means of at least one endoscopic visualization instrument equipped with at least one lens system returning a working image and/or by means of at least one endoscopic surgical instrument equipped with at least one working head capable of carrying out an interaction with a working surface. This interaction can be done for example directly, for example in the form of a blade, or with a working movement such as a removal or a gripping by closing forceps.

These instrument movements are carried out in a working space, delimited by working surfaces within an interior space of a hardware element forming a model called thematic model.

This interior space has internal dimensions of one and the same order of magnitude as the region of interest, typically equal to the real dimensions, or different by a factor smaller than five, or even smaller than two, or even smaller than 1.5. This interior space can have shapes which can be different from or similar (or even identical) to the shapes of the region of interest.

According to the invention, the working image used by the operator shows the interior space of the thematic model. As will become more clearly apparent later, this must be understood in the sense that this working image shows the interior of the model as it exists in reality, i.e. everything that appears there, both the hardware content of the model and the instruments used and any objects manipulated with these instruments.

This method is implemented by at least one electronic or computer device arranged to form a management unit, equipped with a connection interface which is functionally connected to at least the visualization instrument and/or the surgical instrument, and possibly also to the thematic model.

It moreover comprises receiving and processing, by said management unit by means of said connection interface, data called exercise data originating from one or more elements (for example two or three) out of:
the visualization instrument(s),
the surgical instrument, and
the thematic model.

According to the invention, the method moreover comprises a process of evaluating the instrument movements during said exercise, carried out by said management unit and comprising:
processing said exercise data in order to model all or some of the instrument movements, and
comparing said movements with goal data showing one or more determined criteria.

Typically, this process of evaluating an exercise (including monitoring the exercise) is carried out within a process called exercise processing process, which moreover comprises providing instructions to the learning operator, for example by display on the screen of the management unit. These instructions are predetermined, stored and/or calculated in real time. They comprise in particular a report of the goals to be achieved, possibly constraints to be respected and/or additional information or directions, at the start of and/or during the exercise.

As is understood, the context is that of a manual dexterity exercise in the use of hardware instruments, typically without motorization and/or without assistance.

This dexterity uses the haptic sensations returned by the instruments, not only their weight but also the resistance of the materials on which the operator is working.

Unlike methods or appliances for simulation by virtual reality, this method provides a hardware simulation where the haptic feedbacks are real and accurate for the operator, while making it possible to monitor and evaluate the training. The implementation and the development of the method are largely simplified therein, with many fewer sensors and much less image management, while providing a very great haptic realism; even if the visual realism and the progress of the procedure are less accurate than in a virtual reality.

The method thus makes it possible to train the operator on real operating instruments. It also makes it possible to use training instruments which have certain characteristics in common with the operating instruments, for example the weight and the cutting edge or the spring; while potentially being less costly because not subject to other constraints such as corrosion or cleaning.

The invention moreover proposes a certain number of features which can be combined in different ways depending on the teaching aims.

According to a feature, the evaluation process comprises at least a processing of image data originating from the visualization instrument and showing one or more working images, said processing comprising at least:
a graphic recognition process carrying out an identification and/or a dimensional measurement, for example combined with or after manual designation, of one or more target patterns carried by the working surface (for example by recognizing their contour and/or their colours); and
an analysis of said working images so as to evaluate the position and/or the movements of the lens system with respect to the working space.

This analysis comprises for example, for one or more target patterns identified:
evaluation of its centring or of its position within the working image, making it possible for example to evaluate the orientation of the lens system with respect to said pattern and therefore to the working space; and/or
measurement of its apparent dimension from a target pattern within the working image, making it possible to evaluate the distance between the lens system and said pattern.

These items of information are for example combined with each other then compared with data showing the working space, in order to evaluate the position and the exact orientation of the lens system with respect to the model, and to deduce therefrom a conformity evaluation for the exercise.

According to another feature, the evaluation process comprises at least a processing of movement data originating from the visualization instrument, and/or from the surgical instrument or its working head.

These data thus produce an internal detection and show a position and/or a movement of one or more articulations within said instrument, for example by sensors for movements or the position of different articulations within the instrument, and/or by recording commands sent to actuators within the instrument.

According to yet another feature, the evaluation process comprises at least a processing of movement data originating from the surgical instrument.

These data thus produce an internal detection and show a number of working movements carried out by the working head, alone or combined with data showing the completeness or the success of said working movements. These data are provided for example by sensors for detecting movement, or even amplitude or movement or intensity of effort applied, or by recording movement commands, possibly combined with data of contact between the working head and the model or one of its sub-assemblies.

Again according to another feature, the evaluation process comprises at least a processing of interaction data, showing the occurrence of one or more events of interaction, in particular by contact, between at least two elements out of:
at least one surgical instrument (or several) or its working head,
one or more mobile elements forming sub-assemblies of the thematic model, and
one or more detection portions of the thematic model in the case where it is connected to the management unit.

Such interaction events are for example a detection of a contact or near contact, for example by electrical detection of the galvanic or capacitive or inductive type, for example between the working head and a determined portion the model or between two portions of the mobile models with respect to each other, or a detection of the end of such a contact or near contact.

If this is interaction between physical instruments and/or with the hardware interior of a model, the real image of which is used by the operator, it is clear that these are real interactions.

According to another feature, the evaluation process comprises at least a processing of the measurement of a duration for carrying out the exercise, which is calculated between a start point and an end point, which are each determined by a manual activation and/or by a detection of an event by means of data (for example event data, or data resulting from a processing of movement data) received from one or more elements out of:
at least one surgical or visualization instrument (or several) or its lens system or its working head,
one or more mobile elements forming sub-assemblies of the thematic model, and
one or more detection portions of the thematic model in the case where it is connected to the management unit.

Such events are for example a start or an end of movement, a detection of entrance in or exit from the working space, a detection of a determined contact with the model, a detection of a determined working movement possibly validated by subsequent manual input or by calculation, a detection of an end of contact between the model and one of its sub-assemblies.

Overall Evaluation

Preferably, the method comprises a processing, within the management unit, of a plurality of different exercises typically corresponding to a plurality of different thematic modules. Based on said recordings, for example a processing of overall evaluation of a learning process including said exercises is carried out.

Conditional Evolution

Possibly, based on a first evaluation of a first set of exercises (one or more exercises), it moreover comprises an activation and processing, or not, of a second set of exercises (one or more exercises).

System

According to another aspect, the invention also proposes a system for assisting the learning, by an operator, of endoscopic surgery applied to at least one region of interest of the human or animal body, comprising at least one device, called a thematic model, forming a hardware element having an interior space forming a working space and delimited by surfaces called working surfaces.

This interior space has internal dimensions of one and the same order of magnitude as the region of interest, so as to make it possible for said operator to practice a set of instrument movements, called an exercise, within said working space by means of at least one endoscopic visualization instrument equipped with at least one lens system returning a working image and/or an endoscopic surgical instrument equipped with at least one working head capable of carrying out an interaction with the working surface (with or without working movement). This working surface is understood here as comprising the walls, but also the surfaces, of one or more working structures contained in said working space.

According to the invention, this system moreover comprises at least one electronic or computer device arranged to form a management unit, equipped with a connection interface which is functionally connected or connectable to at least the visualization instrument and/or the surgical instrument, and possibly to the thematic model.

Typically, this system is arranged to implement a process as set out here.

Typically, the thematic model comprises on the one hand a support (looking alike or not) forming a manikin or a manikin portion capable of being positioned in a realistic position for a surgical operation of the type concerned; and comprises on the other hand a plurality of different structures, called thematic modules, arranged to each be able to be fixed selectively (and optionally connected) in one and the same location of said support.

In the case of a model having contact or near-contact detection regions, these can be situated in the module or in the support manikin or in both. According to the configurations, the management unit can thus be connected to the interchangeable module, to the support (for example a support head) or to the thematic module or to both.

Each of said thematic modules then comprises an interior space arranged to form a working space once fixed on said support, the assembly formed by the support and said thematic module thus producing the thematic model.

Preferably, the working surface comprises one or more detection regions, which are each arranged to detect a contact or near-contact interaction with the surgical instrument or the visualization instrument, by itself or in cooperation with said instrument, which detection regions are operationally connected or connectable to the management unit.

This interaction is detected for example by a pressure or temperature sensor, or by an electrical circuit capable of detecting an interruption caused by a destruction of or a cut in a portion of the working surface. The cooperation is typically an electrical cooperation, for example by galvanic contact or by capacitive or inductive interaction.

Modules or Models

Different types of thematic modules are proposed by the invention, which can also each be produced integrated in a thematic model.

According to a preferred embodiment, the invention is applied to the learning of endonasal surgery. Typically, the thematic model shows a human head, and the working space is dimensioned to simulate the dimensions of a cavity or portion of the endonasal cavity.

The invention proposes in particular the following configurations and arrangements for a system as set out here, or for a model or module arranged to produce a thematic model or a thematic module within such a system.

In one of these configurations, the invention proposes a thematic model or module which carries, on its working surface, a plurality of target patterns, of one or more determined shapes and/or colours, drawn in different portions of said working surface so as to be visible by the visualization instrument in different positions and/or at different angles, in particular with the management unit is arranged to implement at least one method as set out here.

In another of these configurations, the thematic model or module carries, on its working surface, in particular on at least two lateral surfaces, one or more hooks each capable of receiving a flexible and/or elastic ring having to be put in place by means of the surgical instrument in a form the working head of which carries forceps, in particular with the management unit arranged to implement:
a method as set out here, arranged so as to evaluate a number of movements of the surgical instrument which are necessary for putting in place a determined number of rings on a determined combination of said hook(s), and/or
a method as set out here, arranged so as to evaluate the number of working movements of the forceps which are necessary to put in place a determined number of rings on a determined combination of said hook(s), and/or
a method as set out here, arranged so as to evaluate the number of contacts which are occasioned between the surgical instrument and the working surface before putting in place a determined number of rings on a determined combination of said hook(s).

In another of these configurations, the thematic model or module contains, in its working space, one or more sub-assemblies each formed by an element to be dissected which is fixed to the working surface, and which are intended to be separated from each other along a bonding surface, by means of the surgical instrument in a form the working head of which carries dissecting forceps. In particular with the management unit arranged to implement:
a method as set out here, arranged so as to evaluate a number of movements of the surgical instrument which are necessary for carrying out said separation, and/or
a method as set out here, arranged so as to evaluate the number of working movements of the forceps which are necessary for carrying out said separation.

For example, at least one such element to be dissected comprises at least:
a first element made of foam, in particular a dry foam, which is fixed by one face to the working surface and which has on another face a bond plane, positioned transverse to a direction of introduction of the instruments into the working space, and on the other hand one or more second distinct absorbent elements made of foam (which are typically moistened before the exercise) which are bonded onto said first element along its bond plane, which second elements are intended to be separated from said first element along said bond plane; and/or
one or more elements made of elastomer each simulating a polyp and which is fixed on the working surface; and/or
one or more flexible labels, in particular made of foam, each of which is bonded onto the working surface according to a bonding having a determined adhesive strength, in particular at a rate of several labels which have calibrated adhesive strengths and are different from each other.

In another of these configurations, the thematic model or module has an elongated element, called a metallic path, produced from an electrically conductive material, around which a mobile element, in particular a ring, can slide longitudinally between a starting position and an arrival position by means of the surgical instrument in a form the working head of which carries forceps.

The management unit is then arranged to implement a method as set out here, arranged so as to evaluate the number of contacts which are occasioned between the surgical instrument and the metallic path in order to move the mobile element from the starting position to the arrival position; in particular with the management unit is moreover arranged to implement:
a method as set out here, arranged so as to evaluate a number of movements of the surgical instrument which are necessary for moving the mobile element from the starting position to the arrival position, and/or
a method as set out here, arranged so as to evaluate the number of working movements of the forceps which are necessary for moving the mobile element from the starting position to the arrival position.

In another of these configurations, the thematic model or module has one or more (in particular metallic) conductive edges which surrounds an entrance opening of the working space in order to form a working corridor around the instrument(s); and contains a plurality of receptacles, between which the operator has to move one or more mobile elements, by means of the surgical instrument in a form the working head of which carries forceps.

In particular, the management unit is then arranged to implement a method as set out here, arranged so as to evaluate the number of contacts which are occasioned between the surgical and/or visualization instrument on the one hand and the conductive edges on the other hand in order to move said mobile elements from one receptacle to the other.

In particular, the management unit is moreover arranged to implement:
a method as set out here, arranged so as to evaluate a number of movements of the surgical instrument which are necessary for moving said mobile elements from one receptacle to the other, and/or
a method as set out here, arranged so as to evaluate the number of working movements of the forceps which are necessary for moving said mobile elements from one receptacle to the other.

In another of these configurations, the thematic model or module comprises, inside its working space, a hard-boiled egg having to be dissected in a determined region, in particular which has a marking showing said region to be dissected and/or a start of a crack in its shell.

In another of these configurations, the thematic model or module comprises, inside its working space, one or more compartments, called storage spaces, sealed by a mobile cover plate which is held closed by elastic means, in or from which the operator has to insert or remove one or more mobile elements; in particular with the management unit moreover arranged to implement:
a method as set out here, arranged so as to evaluate a number of movements of the surgical instrument which are necessary for inserting or extracting said mobile elements in or from said storage spaces, and/or
a method as set out here, arranged so as to evaluate the number of working movements of the forceps which are necessary for inserting or extracting said mobile elements in or from said storage spaces.

In another of these configurations, the thematic model or module comprises, inside its working space, one or more compartments each closed by an internal wall pierced by an opening narrower than said internal wall, the operator having to move one or more mobile elements from one compartment to the other.

In particular, the management unit is arranged for:
a method as set out here, arranged so as to evaluate a number of movements of the surgical instrument which are necessary for moving one or more mobile elements from one compartment to the other, and/or
a method as set out here, arranged so as to evaluate the number of working movements of the forceps which are necessary for moving one or more mobile elements from one compartment to the other.

Preferably, the management unit is moreover arranged to implement:
a method as set out here, arranged so as to evaluate the number of contacts which are occasioned between the surgical and/or visualization instrument on the one hand and one or two conductive elements positioned in order to simulate optic nerves, to move one or more mobile elements from one compartment to the other.

In another of these configurations, the thematic model or module comprises, inside its working space, a cavity opening onto a surface pierced by a plurality of orifices, in which one or more mobile elements have to be inserted and fixed by wedging.

In particular, the management unit is arranged for:
a method as set out here, arranged so as to evaluate a number of movements of the surgical instrument which are necessary for inserting said mobile elements in said compartments, and/or
a method as set out here, arranged so as to evaluate the number of working movements of the forceps which are necessary for inserting said mobile elements in said compartments.

Preferably, the management unit is moreover arranged to implement:
a method as set out here, arranged so as to evaluate the number of contacts which are occasioned between the surgical and/or visualization instrument on the one hand and a metal grid, in order to insert said mobile elements in said compartments.

For the different embodiments of the invention, in the form of a method or system or models or modules, the general idea is to propose exercises which are adapted to the level, which are dynamic, fun, and of progressive difficulty, and which make immediate feedback on the different aptitudes tested possible. A key principle is the division of the surgical tasks: it makes it possible to identify the tricky points and to propose a progressive path, in order to make it possible for the learner to familiarize themselves with the different facets of endoscopic surgery. These facets include in particular conceptualizing and carrying out moves in 3D with a vision separated into 2D, learning in terms of force feedback, mastering and carrying out more complex surgery steps, without then with simulation of the bleeding. It moreover makes it possible to work on specific points in an improvement process, and to propose personalized activities, including for experts.

Various embodiments of the invention are provided, integrating the different optional characteristics set out here in all of their possible combinations.

### BRIEF DESCRIPTION OF THE DRAWINGS

Other features and advantages of the invention will become apparent from the detailed description of an embodiment which is in no way limitative, and from the attached drawings, in which:

FIG. 1 is a diagram which illustrates a system according to an embodiment example of the invention;

FIG. 2 is a diagram in a subjective view which illustrates the interior of a thematic module of a first type, with purely visual interaction;

FIG. 3 is a diagram in a subjective view which illustrates the interior of a thematic module of a second type, with movement of mobile elements;

FIG. 4 is a diagram in a subjective view which illustrates the interior of a thematic module of a third type, with dissection of spongy bodies;

FIG. 5 is a diagram in a subjective view which illustrates the interior of a thematic module of a fourth type, with dissection of adhesive labels;

FIG. 6 is a diagram in a subjective view which illustrates the interior of a thematic module of a fifth type, with contactless movement on an imposed trajectory;

FIG. 7 is a perspective diagram which illustrates the interior of a thematic module of a sixth type, with movement of mobile elements between several receptacles and under lateral control;

FIG. 8 is a perspective diagram which illustrates the interior of a thematic module of a seventh type, with dissection of an egg;

FIG. 9 is a perspective diagram which illustrates the interior of a thematic module of an eighth type, with elastic drawers;

FIG. 10 is a perspective diagram which illustrates the interior of a thematic module of a ninth type, with insertion of elements;

FIG. 11 is a diagram which illustrates the interior of a thematic module of a tenth type, with insertion of plugs.

### DESCRIPTION OF EMBODIMENTS

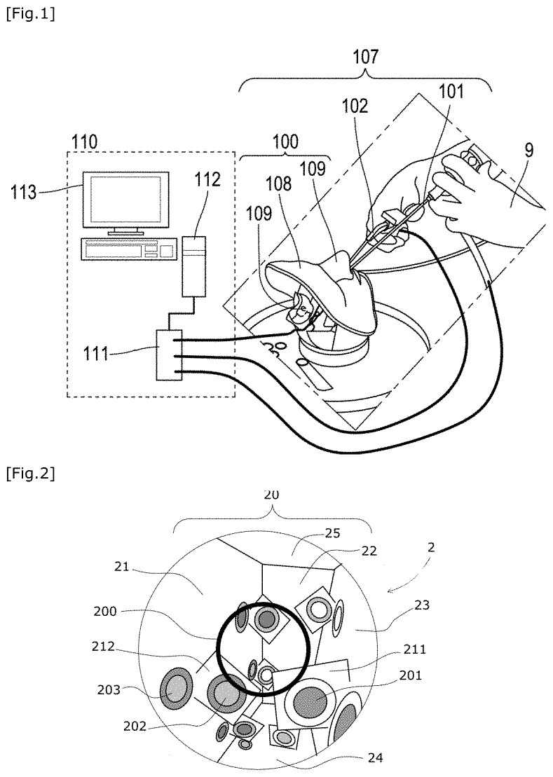

FIG. 1 illustrates an assistance system 1 according to an embodiment example of the invention, which comprises a simulator 107 for endonasal surgery.

This simulator 107 comprises a thematic model 100, a visualization instrument 101 and a surgical instrument 102. It can also comprise several instruments, but only one is shown here. The thematic model 100 comprises a support head 108 which is arranged to make a realistic installation possible, i.e. similar to an installation of a patient in the operating room. This support head 108 is the receptacle of a module 109, interchangeable among a plurality of modules 2 to 11 designed for a teaching progression, and with this module 109 forms a thematic model 100.

The simulator 107 comprises an endoscope 101, here operated with the left (non-dominant) hand of an operator 9. With their right (dominant) hand, this operator operates a surgical instrument 102.

The simulator 107 is connected to management software, which gives the instructions and effects the management of the collection and evaluation of the data, which is executed on a management unit 110, and the functioning of which is described below. This management unit 110 here comprises a computer 112 equipped with a screen 113. Here by means of a connection box 111, the computer and its software are connected to:
the endoscope 101, so as to receive and record the images originating from its lens system,
the surgical instrument(s) 102, and
to the thematic model 107, here by its thematic module 109.

Each thematic model 100 (in its entirety or through its single module 109) corresponds to one or more exercises, i.e. a series of visualization and/or surgical moves which have to be carried out by the operator 9 within the working space of the model 100 (here within the module 109).

Different examples of modules are illustrated in FIG. 2 to FIG. 11, which will be described hereinafter, only in terms of their differences.

In these examples, the interior of the thematic module 109 has a substantially parallelepiped shape, with an opening on a small face featuring the nostrils. This space is delimited, according to the frame of reference of the patient, by right-hand 21 and left-hand 23 lateral vertical walls, a bottom wall 22, and lower 24 and upper 25 walls. Typically, these walls are rigid in the portion forming the internal portion the endonasal cavity; whereas they are flexible in their portions delimiting the opening, on the face featuring the nostrils. This flexibility thus makes a slight deformation possible for the passage of the instruments, similarly to reality.

The modules described can, however, be produced with shapes different from a parallelepiped, for example cylindrical, and preferably while keeping an elongated dimension are an end includes the nostrils and bears the access opening for the instruments.

For example for other types of surgery, similar modules can be used with the same devices and interior structures, even if the shape of the working space differs from the examples presented here.

“Target” Module—Surgical Site

FIG. 2 illustrates the interior of a thematic module 2 of a first type, also called “Target”, as seen through the endoscope 101.

The module 2 has a working space 20, delimited in particular by three main surfaces: two lateral surfaces 21 and 23, and one distal surface 22.

In endonasal surgery, the first difficulty is to master the movements within the narrow surgical site that is the nasal cavity, and to know how to correctly reveal what it is desired to see; centred image, sufficient zoom, stable image. It should also comprise the three-dimensional vision, to be reconstructed mentally based on the 2D information visible on the screen and the depth information given by the proprioception of the non-dominant hand guiding the lens system. With this aim in mind, the Target module shows coloured and identified targets, arranged on the three faces 21, 22, 23 of the module 2.

In this example, approximately 14 targets are visible, labelled 201, 202, 203 for the closest. These targets have distinct combinations of patterns and/or colours, here combined between their circular interior and their peripheral contour.

It will be noted that certain targets 201, 202 are applied to working surfaces 211, 212 which form projections on the three main faces 21, 22, 23. This gives these targets different orientations, which each require a different position of the lens system in order to visualize them correctly and to validate them.

A calibrated ring 201 is integrated in the video, in the centre of the viewing area, by the software. In order to process and validate a target, the learner has to obtain a centred vision of the target and adapt the distance of their lens system (zoom) in order that the target occupies the whole surface of the disc delimited by the ring on the screen. The target is considered to be validated once the image has been stabilized and validated by the automatic recognition of the software, here based on the recognition of their combinations of patterns and colours.

The learner has to pass from one target to the other according to a sequence announced by the software, and validate each target using the software before passing to the next.

This module of targets can be combined with all of the other modules, for example by adding, into the exercises of the other modules, targets to be validated similarly, in which the evaluation will then include these validations.

“Rubber Band” Module—Coordination—Precision

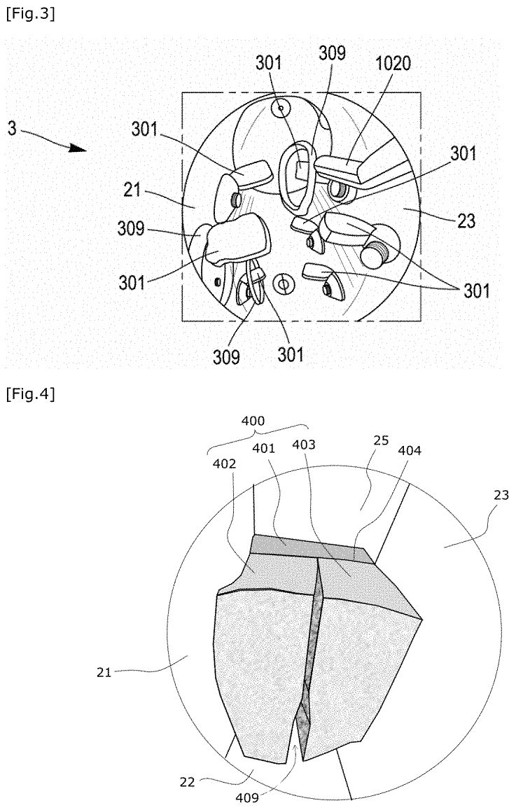

FIG. 3 illustrates the interior of a thematic module 3 of a second type, with movement of mobile elements.

Once mastery of the surgical site has been obtained, an additional difficulty is added by the need to coordinate the video tool 101, carried by the non-dominant hand, with the surgical instrument 102 carried by the dominant hand. As this surgical instrument 101 can be blunt or sharp, it must not be lost from view during its movements in the nasal cavity. Finally, the precise use of this instrument 101 will ensure a time saving (no repetition of ineffective moves) and a dissection safety (gentle and precise movements).

With this aim in mind, this teaching module 3 that has been created is equipped with different hooks 301 on the two lateral faces 21, 23, and which are here arranged in two rows of two hooks in each. The eight hooks 301. Four are equipped at the start with coloured elastic rings 309, which the learner will have to move with Blakesley forceps (forming the working head 1020) in an announced sequence, without dropping them and without stablishing electrical contact between the forceps 101 and the hooks 301.

The openings of the forceps 1020 (constituting working movements) and the movements of the instrument 101 are detected and recorded by communication of the management unit 110 with the surgical instrument 101. The contacts with the hooks are detected and recorded by communication of the management unit with the surgical instrument 101 and with the hooks 301 of the module 3.

“Sponge” Module—Dissection

FIG. 4 illustrates the interior of a thematic module 4 of a third type with dissection of spongy bodies.

Like open dissection, endonasal dissection is based on the recognition of the dissection planes and respect of the structures. These two points are influenced by the abilities of the operator to produce a quality image (worked on with the Target module) and to keep control of their surgical instrument: precision of the grip, control of the traction forces applied in order to avoid rupture of the tissue (Rubber band module).

This teaching module 4 aims to combine these points. It involves “dissecting” a household sponge of the bi-material type, by detaching the absorbent yellow portion from the abrasive green portion. For this, a rectangular piece of sponge 400, here graded as 3.5×2.5 cm, is fixed by its abrasive layer 401 in a space arranged in the upper wall 25 of the interior space of the module 4. In advance, the absorbent portion is moistened and divided 409 according to a median sagittal plane 200 in order to differentiate between the right-hand 402 and left-hand 403 sides. The aim for the learner is to dissect each yellow portion 402, 403 from its green support 401, while respecting the bond plane 404 between them and without tearing the yellow portions.

The exercise is carried out for example with dissecting forceps (not visible here), and the movements and openings of the forceps of the surgical instrument are recorded.

“Polyps” Module—Dissection

A thematic module not illustrated here contains pseudopolyps, produced from an elastomer having a realistic texture. The module is standardized in order to comprise pseudopolyps of several determined types. The aim for the learner is to dissect and remove each polyp. Movements and openings of forceps are recorded.

“Sticky” Module—Dissection

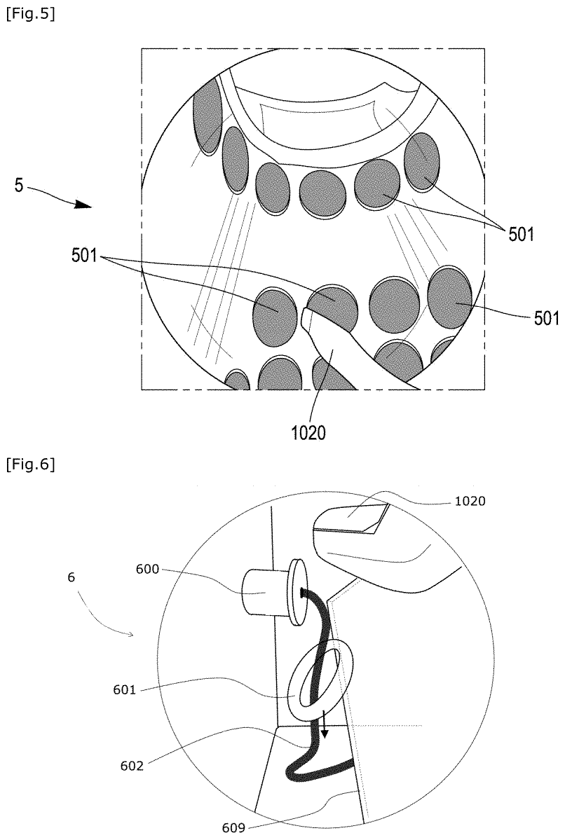

FIG. 5 illustrates the interior of a thematic module 5 of a fourth type, with dissection of adhesive labels.

The cavity here has a substantially cylindrical or rounded shape, but can also be produced in a parallelepiped shape.

A module 5 containing self-adhesive labels 501, in particular made of elastomeric foam, is inserted in the support head 108. These labels 501 are bonded onto the working surface(s), and are chosen with a calibrated adhesive strength to the underlying plane. The aim for the learner is to detach these labels 501 and remove them from the working surface. The movements and openings of forceps are recorded.

“Ring” Module—Use of the Lens System to Push Back Obstacles and for Fine Manipulation

FIG. 6 illustrates the interior of a thematic module 6 of a fifth type, with contactless movement on an imposed trajectory.

This module contains a metallic path, here a curved guide rod 601 which is fixed to the walls at its end(s), around which a ring 602 runs. Electrical contact between the forceps 1020 and the guide 601 is detected by communication between the surgical instrument 101 and the guide 601, and is recorded as an error. Obstacles, such as a mobile partial partition 609, are positioned such that the learner is obliged to push them back with the aid of the lens system of the visualization instrument 101. These obstacles are positioned in front of the path, i.e. in the front portion of the cavity, so as to mask the path 601 in full or in part when it is accessed from the opening. They can be inserted or removed before the exercise, according to the desired difficulty. Movements, openings of the forceps 1020 and contact of the forceps 1020 with the metallic path 601 are recorded. This type of obstacle can also be provided in other modules, such as the other modules described here.

“Electrocute” Module—Mastery of the Instrument in its Working Corridor

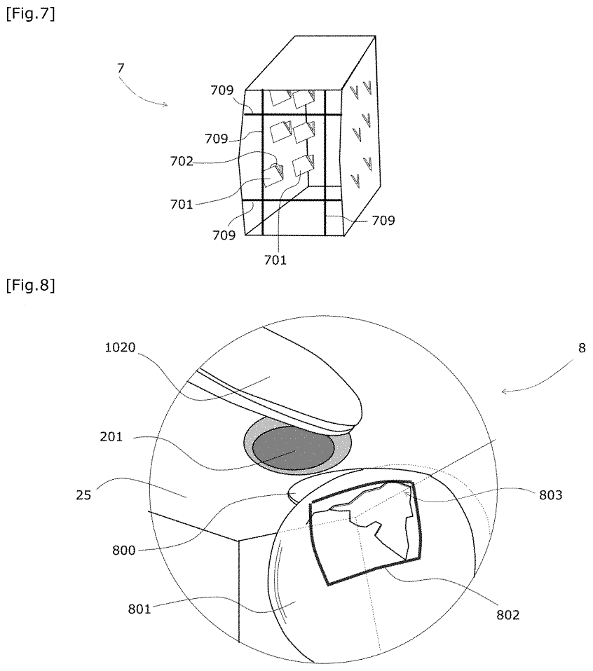

FIG. 7 illustrates the interior of a thematic module of a sixth type, with movement of mobile elements between several receptacles with control of the lateral movements.

This module consists of moving mobile, in particular metallic, elements 702 from one receptacle 701 to the other 701 without electrical contact between the forceps and metallic wires 709 which pass through the front portion of the working space. As an option, the receptacles are combined with targets, which have to be processed and validated as described previously. It is also possible to add obstacles as presented previously.

In particular, the movements and openings of the forceps 1020 are recorded, as are the contacts of the surgical instrument 102 with the wires 809 which form the periphery of a working corridor, as well as the detections and validations of the targets.

“Egg” Module—Dissection or Milling Under a Microscope

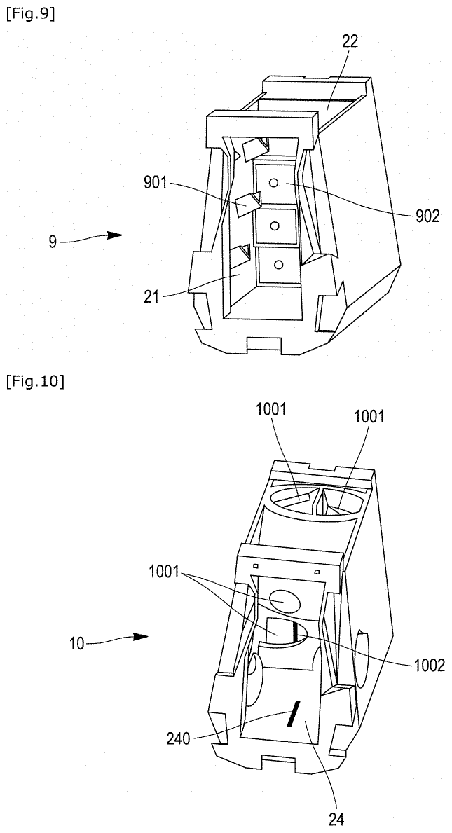

FIG. 8 illustrates the interior of a thematic module 8 of a seventh type, with dissection of an egg.

This module has a space 800 arranged in the upper wall 25 in order to be able to fix a hard egg 801 there with its shell. The hard egg is in fact a tool already validated as realistic in several simulation scenarios. This is the case for learning dissection or milling under a microscope. A calibrated rectangle 802, for example of 3 cm², is drawn in advance with a marker pen on the portion of the egg hanging over into the cavity.

In a first portion of the work of the exercise, this rectangle 802 has to form the subject of a milling under the conditions reproducing those of the surgical site, for example with a “handpiece”, i.e. a motorized tool held in the hand and which is specific to endonasal surgery.

In an alternative version without milling, the upper left-hand corner 803 (patient reference) is impacted before the exercise in order to initiate the dissection. The aim is then to dissect the shell in all of this rectangular surface, which is necessary and sufficient, without cutting into the egg white. This portion of the work is only finished when the whole surface drawn on has been removed, and for example only on this surface.

A second portion of the work of the exercise consists of dissecting the egg white without cutting into the yolk.

Movements of lens system, and forceps, and openings of the forceps are recorded. Optionally, data indicating whether or not there was injury to the portions of the egg that are to be avoided are entered.

“Clean Up Now” Module—Four-Hands Surgery

FIG. 9 illustrates the interior of a thematic module 9 of an eighth type, with a compartment including elastic drawers. This module comprises several spaces in which mobile objects can be inserted. These objects are moved as a function of the instructions from the software.

These spaces here comprise gutters 901 which protrude transversely from a wall 21, for example closed gutters or, as here, gutters which are open and inclined in a direction directed upwards. They also comprise six drawers 902, the mobile portions of which are brought back towards the wall 22 by an elastic system which obliges the user to have the help of another operator, thus making it possible to train for four-hands surgery. Movements and openings of forceps are recorded.

“Rough Diamond” Module

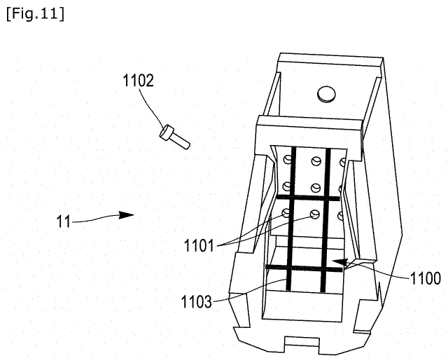

FIG. 10 illustrates the interior of a thematic module 10 of a ninth type, with insertion of elements.

This module comprises several internal spaces 1001 forming sub-assemblies of the working space, and in which mobile objects can be inserted (pearls, diamonds, counters, etc.). These compartments are closed by an internal wall pierced by one or more openings narrower than said internal wall. Here, these internal walls are substantially vertical, and their opening is higher than the floor of their compartment, thus obliging the operator to lift the mobile object in order to extract it from said compartment. The objects are moved as a function of the instructions from the software. Obstacles and a central partition can be inserted at will, for example in a slit 240 provided in a wall, here the lower wall 24. Two conductive elements, here vertical metal bars 1002, simulate the presence of the optic nerves. Movements, openings of the forceps and any contacts with the optic nerves are recorded. Targets (not visible here) are also arranged in the sphenoid cavity, which contains the optic nerves 1002, and have to be aimed at by the operator in order to be detected and validated by the software.

“TicTacToe” Module

FIG. 11 illustrates the interior of a thematic module 11 of a tenth type, with insertion of plugs.

This module comprises spaces 1101 for inserting coloured pins 1102, for example in the form of nails. The insertion of the coloured pin(s) 1102 in their dedicated holes 1101 makes it possible to create a target recognizable by the software, for example by its pattern and its colours, which thus validates the success of the step.

It is also possible to use this module in a classic game of noughts and crosses, with two operators facing each other. A metal grid 1103 is arranged in front of the cavity 1100: it acts as an obstacle which must not be touched. Movements, openings of the forceps and any contacts with the grid are recorded.

“Anatomic” Modules

Different types of “anatomic” modules are also provided, not illustrated here. This module realistically reproduces the nasal anatomy, with the colours and/or textures corresponding to reality. It can be standardized or personalized, developed specifically based on a scan. It can be in one or more parts.

Bleeding Function

Each of the different modules can be produced with a bleeding function, in order to increase the complexity thereof. The bleeding is simulated thanks to a pump, controlled for example manually or by the management unit. It can be controlled to be continuous or pulsating. It can be permanent or triggered, for example during a contact with a detection region and/or as a function of time.

This function is provided in particular as a supplement within the “egg”, “dissection” and “diamond” modules. It can be activated or deactivated by the software, in the programming of the exercise or manually. The inlet of the bleeding can be positioned on the lateral, upper or back walls of the modules. For the “egg”, “dissection” and “diamond” modules, this inlet is placed in the same places as in the anatomical modules, where the positioning reproduces that of the ethmoidal (upper wall) and sphenopalatine (lateral walls) arteries.

This function also makes it possible to simulate a biological liquid under pressure (such as the cerebrospinal fluid). For example for endonasal surgery, this makes it possible to effect exercises consisting of plugging holes through which this liquid flows under pressure.

Management Unit

In the management unit 110, the software application has a graphic interface making it possible to display all of the information necessary for the success of the exercises: the different instructions, the progression of the exercise, the video feedback from the camera 101 in order to see into the nasal cavity, etc. This application can be used in particular by two types of user: the students, who have access to all of the exercises and can launch them; and the administrators, these will be for example professors or surgeons, who will be able to do everything that the students can, but additionally have the possibility of creating the exercises, the programs and the sessions.

Each session can unite one or more programs of one or more exercises. The exercises are graded by level as a function of how difficult they are to carry out.

When an administrator desires to create an exercise, they open in front a detailed window which makes it possible for them to fill in a certain number of fields: the name of the exercise, the general description, its type, etc. In addition to this general information, it is possible to tick certain additional options (bleeding, target, etc.). An important part in the creation of an exercise is the choice of the measurements and calculations that it is desired to effect, as well as the instructions that the user will have to follow.

For the user, once the exercise has been chosen, a new window opens showing the interface of the exercise. In the centre a frame which displays the video depiction, on the left-hand portion the description of the exercise and the instructions, above it the current instruction and at the top right a progress bar showing the progress in the exercise. The user then has several possibilities: start the exercise, launch a video demonstration of the exercise, or also stop the exercise.

Once the exercise has been launched, the user merely has to follow the instructions written on the screen and carry out the exercise. At the end of this, they can save the video recording of the exercise. Finally, the page of the scores is displayed, the learner can then visualize their score on each measurement that was selected for the exercise and finally their overall score for the whole of the exercise.

The scores of the exercises are evaluated as a function of different criteria, which derive from or are calculated by the computer based on the data received from the simulator 107 by the management unit 110. These criteria are chosen and programmed during the definition of the exercise. By way of example, the system 1 can use, according to the exercises, all or some of the following criteria:
measurement of the time necessary for carrying out the exercise,
number of times the forceps are opened/closed,
contacts with detecting portions,
centring on the targets,
movements of the lens system,
movements of the instruments.

The movements make it possible for example to evaluate the stability and orientation of the instruments.

Other criteria can be entered manually, and recorded manually or by programming, for example a number of impacts in the egg, a number of sponge fragments obtained after dissection, etc.

The majority of the criteria correspond directly to an objective evaluation, calculated by the management unit. Sub-scores can be combined with different weightings in order to obtain a final score. These criteria make it possible to carry out a real path on several levels, for each user, with thresholds per level which make it possible to access the higher level. The thresholds are regulated by the administrators as a function of the goal aimed at and the level of the learner. As long as the threshold has not been crossed, the learner has to continue to practice and cannot attempt the exercises of the higher levels, unless the administrator effects a manual validation, signifying for example that the level has been acquired.

1.-19. (canceled)

20. A method for assisting the learning, by an operator, of endoscopic surgery applied to at least one region of interest of the human or animal body, in particular an endonasal region, during an exercise carried out by said operator,
said exercise comprising a set of movements of instruments operated by hand, carried out by said operator, by means of at least one endoscopic visualization instrument equipped with at least one lens system returning a working image and by means of at least one endoscopic surgical instrument equipped with at least one working head capable of carrying out an interaction with a working surface,
said instrument movements being carried out in a working space, delimited by working surfaces within an interior space of a hardware element forming a model called thematic model;
said interior space having internal dimensions of one and the same order of magnitude as the region of interest;
said method comprising the working image used by the operator is a camera video feedback and shows the interior space of the hardware element forming said thematic model, each thematic model corresponding to one or more exercises, comprising a series of visualization and/or surgical moves which have to be carried out by the operator;
and said method is implemented by at least one electronic or computer device arranged to form a management unit, equipped with a connection interface which is functionally connected to at least the visualization instrument and/or the surgical instrument, and to the thematic model;
comprises receiving and processing, by said management unit by means of said connection interface, data called exercise data originating
the visualization instrument,
the surgical instrument,
the thematic model;
a sub-assembly of the thematic model, said sub-assembly comprising one or more mobile elements;
and said comprises a process of evaluating the instrument movements during said exercise, carried out by said management unit and comprising:
processing said exercise data in order to model all or some of the instrument movements; and
comparing said movements with goal data showing one or more determined criteria; and
the evaluation process comprises at least a processing of interaction data, showing the occurrence of one or more events of interaction between at least two elements out of:
said at least one surgical instrument or a working head with which said surgical instrument is equipped;
one or more mobile elements forming said sub-assemblies of the thematic model; and
one or more detection portions of the thematic model connected to the management unit;
said method providing a hardware simulation where haptic feedbacks are real and accurate for the operator.

21. The method according to claim 20, characterized in that the evaluation process comprises at least a processing of image data originating from the visualization instrument and showing one or more working images, said processing comprising at least:
a graphic recognition process carrying out an identification and/or a dimensional measurement of one or more target patterns carried by the working surface; and
an analysis of said working images so as to evaluate the position and/or the movements of the lens system with respect to the working space.

22. The method according to claim 20, characterized in that the evaluation process comprises at least a processing of movement data originating from the visualization instrument, and/or from the surgical instrument or its working head, which data show a position and/or a movement of one or more articulations within said instrument.

23. The method according to claim 20, characterized in that the evaluation process comprises at least a processing of movement data originating from the surgical instrument, which data show a number of working movements carried out by the working head, alone or combined with data showing the completeness or the success of said working movements.

24. The method according to claim 20, characterized in that the evaluation process comprises at least a processing of the measurement of a duration for carrying out the exercise,
which is calculated between a start point and an end point, which are each determined by a manual activation and/or by a detection of an event by means of data received from one or more elements out of:
at least one instrument or its working head;
one or more mobile elements forming sub-assemblies of the thematic model; and
one or more detection portions of the thematic model in the case where it is connected to the management unit.

25. A system for assisting the learning, by an operator, of endoscopic surgery applied to at least one region of interest of the human or animal body, comprising: at least one device, called a thematic model, forming a hardware element having an interior space forming a working space and delimited by surfaces called working surfaces;
said interior space having internal dimensions of one and the same order of magnitude as the region of interest, so as to make it possible for said operator to practice a set of instrument movements, called an exercise, within said working space by means of at least one endoscopic visualization instrument equipped with at least one lens system returning a working image and/or an endoscopic surgical instrument equipped with at least one working head capable of carrying out an interaction with the working surface;
at least one electronic or computer device arranged to form a management unit, equipped with a connection interface which is functionally connected or connectable to at least the visualization instrument and the surgical instrument, and to the thematic model;
the working surface comprises one or more detection regions, which are each arranged to detect a contact or near-contact interaction with the surgical instrument or the visualization instrument, by itself or in cooperation with said instrument, which detection regions are operationally connected or connectable to the management unit; the thematic model or module contains, in its working space, one or more sub-assemblies, each comprising one or more mobile elements, capable of transmitting data called exercise data to said management unit by means of said connection interface; and
the thematic model or module is arranged to implement a method according to claim 20.

26. The system according to claim 25, characterized in that the thematic model comprises on the one hand a support forming a manikin or a manikin portion capable of being positioned in a realistic position for a surgical operation of the type concerned;
and comprises on the other hand a plurality of different structures, called thematic modules, arranged to each be able to be fixed selectively in one and the same location of said support;
each of said thematic modules comprising an interior space arranged to form a working space once fixed on said support, the assembly formed by the support and said thematic module thus producing the thematic model.

27. The system according to claim 25, characterized in that the thematic model or module carries, on its working surface, a plurality of target patterns, of one or more determined shapes and/or colours, indicated in different portions of said working surface so as to be visible by the visualization instrument in different positions and/or at different angles.

28. The system according to claim 25, characterized in that the thematic model or module carries, on its working surface, in particular on at least two lateral surfaces, one or more hooks each capable of receiving a flexible and/or elastic ring having to be put in place by means of the surgical instrument in a form the working head of which carries forceps, in particular with the management unit arranged to implement:
a method to evaluate a number of movements of the surgical instrument which are necessary for putting in place a determined number of rings on a determined combination of said hook(s), and/or
a method to evaluate the number of working movements of the forceps which are necessary for putting in place a determined number of rings on a determined combination of said hook(s), and/or
a method to evaluate the number of contacts which are occasioned between the surgical instrument and the working surface before putting in place a determined number of rings on a determined combination of said hook(s).

29. The system according to claim 25, characterized in that the thematic model or module contains, in its working space, one or more sub-assemblies each formed by an element to be dissected which is fixed to the working surface, and which are intended to be separated from each other along a bonding surface, by means of the surgical instrument in a form the working head of which carries dissection forceps, in particular with the management unit arranged to implement:
a method to evaluate a number of movements of the surgical instrument which are necessary for carrying out said separation, and/or
a method to evaluate the number of working movements of a forceps which are necessary for carrying out said separation.

30. The system according to claim 29, characterized in that at least one element to be dissected comprises at least:
a first element made of foam which is fixed by one face to the working surface and which has on another face a bond plane, positioned transverse to a direction of introduction of the instruments into the working space, and on the other hand one or more second distinct absorbent elements made of foam which are bonded onto said first element along its bond plane, which second elements are intended to be separated from said first element along said bond plane; and/or
one or more elements made of elastomer each simulating a polyp and which is fixed on the working surface; and/or
one or more flexible labels, in particular made of foam, each of which is bonded onto the working surface according to a bonding having a determined adhesive strength, in particular at a rate of several labels which have calibrated adhesive strengths and are different from each other.

31. The system according to claim 25, characterized in that the thematic model or module has an elongated element, called a metallic path, produced from an electrically conductive material, around which a mobile element, in particular a ring, can slide longitudinally between a starting position and an arrival position by means of the surgical instrument in a form the working head of which carries forceps;
with the management unit arranged to implement a method to evaluate the number of contacts which are occasioned between the surgical instrument and the metallic path in order to move the mobile element from the starting position to the arrival position;
in particular with the management unit moreover arranged to implement:
a method to evaluate a number of movements of the surgical instrument which are necessary for moving the mobile element from the starting position to the arrival position, and/or
a method to evaluate the number of working movements of the forceps which are necessary for moving the mobile element from the starting position to the arrival position.

32. The system according to claim 25, characterized in that the thematic model or module has one or more conductive edges which surrounds an entrance opening of the working space in order to form a working corridor around the instrument(s);
and contains a plurality of receptacles, between which the operator has to move one or more mobile elements, by means of the surgical instrument in a form the working head of which carries forceps;
with the management unit arranged to implement a method to evaluate the number of contacts which are occasioned between the surgical and/or visualization instrument on the one hand and the conductive edges on the other hand in order to move said mobile elements from one receptacle to the other;
in particular with the management unit moreover arranged to implement:
a method to evaluate a number of movements of the surgical instrument which are necessary for moving said mobile elements from one receptacle to the other, and/or
a method to evaluate the number of working movements of the forceps which are necessary for moving said mobile elements from one receptacle to the other.

33. The system according to claim 25, characterized in that the thematic model or module comprises, inside its working space, a hard-boiled egg having to be dissected in a determined region, in particular which has a marking showing said region to be dissected and/or a start of a crack in its shell.

34. The system according to claim 25, characterized in that the thematic model or module comprises, inside its working space, one or more compartments, called storage spaces, sealed by a mobile cover plate which is held closed by elastic means, in or from which the operator has to insert or remove one or more mobile elements, in particular with the management unit moreover arranged to implement:
a method to evaluate a number of movements of the surgical instrument which are necessary for inserting or extracting said mobile elements in or from said storage spaces, and/or
a method to evaluate the number of working movements of a forceps which are necessary for inserting or extracting said mobile elements in or from said storage spaces.

35. The system according to claim 25, characterized in that the thematic model or module comprises, inside its working space, one or more compartments each closed by an internal wall pierced by an opening narrower than said internal wall, the operator having to move one or more mobile elements from one compartment to the other,
in particular with the management unit arranged for:
a method to evaluate a number of movements of the surgical instrument which are necessary for moving one or more mobile elements from one compartment to the other, and/or
a method to evaluate the number of working movements of a forceps which are necessary for moving one or more mobile elements from one compartment to the other;
in particular with the management unit moreover arranged to implement:
a method to evaluate the number of contacts which are occasioned between the surgical and/or visualization instrument on the one hand and one or two conductive elements positioned in order to simulate optic nerves, to move one or more mobile elements from one compartment to the other.

36. The system according to claim 25, characterized in that the thematic model or module comprises, inside its working space, a cavity opening onto a surface pierced by a plurality of orifices, in which one or more mobile elements have to be inserted and fixed by wedging;
with the management unit arranged for
a method to evaluate a number of movements of the surgical instrument which are necessary for inserting said mobile elements in compartments, and/or
a method to evaluate the number of working movements of a forceps which are necessary for inserting said mobile elements in said compartments;
in particular with the management unit moreover arranged to implement:
a method to evaluate the number of contacts which are occasioned between the surgical and/or visualization instrument on the one hand and a metal grid, in order to insert said mobile elements in said compartments.
